SEQUENCE LISTING (2 sequences)

SEQ ID NO: 1 - DNA, 20 bp, Homo sapiens
gcaaatgaaagctttggacc

SEQ ID NO: 2 - DNA, 22 bp, Homo sapiens
atcgtaacattatagactccat

(19) United States
(12) Patent Application Publication
(10) Pub. No.: US 2004/0092475 A1
(43) Pub. Date: May 13, 2004

(54) Organic compounds

(76) Inventors: Sau Wei Li, Horsham (GB); Christopher Terence Poll, Horsham (GB); John Westwick, Horsham (GB); Martin Gosling, Horsham (GB)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/629,190

(22) Filed: Jul. 28, 2003

Related U.S. Application Data
(60) Provisional application No. 60/415,124, filed on Sep. 30, 2002.

(30) Foreign Application Priority Data
Jul. 29, 2002 (GB) 0217503.2

(51) Int. Cl. A61K 48/00; C12Q 1/68; G01N 33/53; G01N 33/567; A61K 39/395; A61K 31/4164

(52) U.S. Cl. 514/44; 424/130.1; 514/396; 435/6; 435/7.2

(57) ABSTRACT

The invention concerns a method of identifying a substance suitable for use in the treatment of a leukocyte-associated inflammatory disease which modulates the activity of a polypeptide encoded by the human transient receptor potential 6 gene. The method involves combining a candidate substance with the polypeptide and measuring the effect of the candidate substance on the activity of the polypeptide.

The present invention relates to the identification of substances or agents that modulate the activity of the transient receptor potential 6 ion channel, TRPC6, and the use of such substances in the treatment of inflammatory diseases, particularly those of the respiratory system.

A variety of cells are attracted into tissues during inflammation. These include various leukocytes, particularly inflammatory phagocytes such as neutrophilic and eosinophilic granulocytes and monocytes. Neutrophils have been associated with inflammation and tissue destruction in respiratory diseases such as chronic obstructive pulmonary disease (COPD), including chronic bronchitis and emphysema associated therewith, and adult respiratory distress syndrome (ARDS), inflammatory bowel diseases such as Crohn's disease and ulcerative colitis, and rheumatoid arthritis. Eosinophils have been associated with respiratory diseases such as asthma and allergic rhinitis.

Critical steps in the action of leukocytes in inflammatory conditions include the migration of these cells into the tissues, e.g. into the airways in respiratory inflammations or to the joints in rheumatoid arthritis, cell activation and the release of a range of inflammatory mediators, leukotrienes, oxygen radicals, proteases. Signals that are needed for leukocyte migration and activation are often communicated through receptors that respond to an increase in the level of cytosolic calcium. Ins(1,4,5)P3 receptors are known to release calcium ions from intracellular stores but less is known about the channels in the plasma membrane through which those ions pass.

The transient receptor potential or “trp” gene of Drosophila is known to encode a Ca⁺⁺ selective store operated Ca⁺⁺ entry (SOC) and this was used to find the human homologue (TRPC) and related genes. This led to the discovery of a family of proteins that function as ion channels i.e. TRPC1-6.

Boulay et al cloned and sequenced murine TRPC6 and described that in “Cloning and expression of a novel mammalian homologue of Drosophila transient receptor potential (trp) involved in calcium entry secondary to activation of receptors coupled by the G_{q} class of G protein” J. Biol. Chem. Vol. 272, 29672-29680 (1997). Murine TRPC6 is available from GenBank under accession number NP_{—}038866.

Human TRPC6, also known as classical TRP6 or TRPC6, is available from GenBank under accession number NP_{—}004612.

D'Esposito et al published the sequence of the human TRPC6 gene in the “Identification and assignment of the human transient receptor potential channel 6 gene TRPC6 to chromosome 11q21-->q22” Cytogenet. Cell Genet. 83 (1-2), 46-47 (1998).

Hofmann et al showed in “Direct activation of human TRPC6 and TRPC3 channels by diacylglycerol”, Nature Vol.397, 21 Jan. 1999 that histamine stimulated greater Ca⁺⁺ influx in CHO-K1 cells if they heterologously expressed a functional TRPC6 channel (demonstrated electrophysiologically). They also demonstrated that TRPC6 channel activity could be measured as Na⁺ influx.

TRPC6 messenger RNA has now been found in blood-derived primary human leukocytes including neutrophils, eosinophils and lymphocytes by reverse-transcription polymerase chain reaction (RT-PCR). This indicates that TRPC6 is present in the cells that are attracted into tissues during inflammation.

It is proposed in accordance with the present invention that substances that attenuate the activation of leukocytes such as neutrophils or eosinophils by inhibiting the influx of calcium ions into such cells are useful for treating respiratory diseases such as asthma and chronic obstructive pulmonary disease. The present invention thus provides TRPC6 as a therapeutic target for such diseases and a “screening assay” for identifying modulators i.e., candidate compounds or agents including peptides, peptidomimetics, small molecules or other drugs, which stimulate or inhibit the activity of the channel formed by the gene product of human TRPC6.

Accordingly, in a first aspect the present invention relates to a method of identifying a substance suitable for use in the treatment of a leukocyte-associated inflammatory disease which modulates the activity of a polypeptide encoded by the human transient receptor potential 6 gene (TRPC6), wherein the method comprises combining a candidate substance with said polypeptide and measuring the effect of the candidate substance on the activity of said polypeptide.

In a second aspect the present invention relates to a pharmaceutical composition comprising a compound that inhibits the influx of calcium ions through a human TRPC6 ion channel and a pharmaceutically acceptable carrier.

In a third aspect the present invention relates to a method of treating a leukocyte-associated inflammatory disease in a subject in need of such treatment, which comprises administering to said subject an effective amount of an antibody which is immunoreactive with a polypeptide encoded by the human TRPC6 gene or an antisense oligonucleotide comprising a nucleotide sequence complementary to a polynucleotide comprising a nucleotide sequence encoding that polypeptide.

In a fourth aspect the present invention relates to a method of treating a leukocyte-associated inflammatory disease in a subject in need of such treatment, which comprises administering to said subject an effective amount of a TRPC6 inhibitor.

As mentioned above, in a first aspect the present invention relates to a method of the present invention for identifying a substance suitable for use in the treatment of a leukocyte-associated inflammatory disease which modulates the activity of a polypeptide encoded by the human transient receptor potential 6 gene. In broad terms this method, or assay, comprises combining a candidate substance with a polypeptide encoded by the transient receptor potential channel 6 gene and measuring the effect of the candidate substance on the activity of that polypeptide.

Substances that are suitable for use in the treatment of a leukocyte-associated inflammatory disease will tend to be enhancers (herein “human TRPC6 agonists”) or inhibitors (herein “human TRPC6 antagonists”) of its activity.

The activity of a TRPC6 gene product, i.e. the polypeptide that forms the TRPC6 ion channel, may be measured, for example, by a cell-based method or screening assay that identifies compounds which have a stimulatory or inhibitory effect on the activity of the human TRPC6 channel, or by an appropriate reporter gene assay.

The abovementioned screening method may be carried out, for example, by preparing cells which express the TRPC6 polypeptide on their surfaces, e.g. insect, mammal or yeast cells and then incubating the resulting cells with the candidate substance to determine the enhancement or inhibition of a functional activity of the TRPC6 polypeptide.

In a suitable test for any activation of the TRPC6 channel, the candidate substance is combined with cells that are stably transfected with TRPC6 and express a functional TRPC6 channel and membrane depolarisation is measured to indicate any TRPC6-mediated Na⁺ influx.

In a suitable test for any inhibition of the TRPC6 channel, cells that express an endogenous G protein-coupled calcium mobilising receptor e.g. muscarinic acetylcholine receptor that can activate TRPC6 channels are used and a receptor agonist e.g. muscarinic acetylcholine receptor agonist is added to stimulate the TRPC6 channels. The cells are treated with the candidate substance prior to addition of the receptor agonist. That should produce an increase in fluorescence, but the magnitude of the increase will be reduced if the candidate substance inhibits the TRPC6 channels. Any change in fluorescence can be measured using suitable equipment, for example a fluorescence imaging plate reader.

Using such methods it has been found that 1-[b-[3-(4-methoxyphenyl)propoxy]-4-methoxyphenethyl]-1H-imidazole.HCl inhibits the activity of the human TRPC6 ion channel.

1-[b-[3-(4-Methoxyphenyl)propoxy]-4-methoxyphenethyl]-1H-imidazole.HCl was disclosed by Merritt et al in Biochem. J. (1990) 271: 515-522 to inhibit receptor-mediated calcium entry (RMCE). It was also disclosed by Inoue et al in Circ. Res. (2001) 88: 325-332 to block the murine TRPC6 ion channel.

The present invention also relates to a pharmaceutical composition that comprises a compound that inhibits the influx of calcium ions through a TRPC6 channel, such as 1-[b-[3-(4-methoxyphenyl)propoxy]-4-methoxyphenethyl]-1H-imidazole.HCl, and a pharmaceutically acceptable carrier.

One can use an antibody which is immunoreactive with the polypeptide encoded by the human TRPC6 gene (herein a “human TRPC6 antibody”) or an antisense oligonucleotide comprising a nucleotide sequence complementary to the polynucleotide comprising a nucleotide sequence encoding that polypeptide (herein a “human TRPC6 antisense oligonucleotide”), to prepare pharmaceuticals that inhibit the accumulation of leukocytes in human tissue.

The aforementioned human TRPC6 antibodies and antisense oligonucleotides may be used to treat leukocyte-associated inflammatory diseases.

Human TRPC6 agonists, human TRPC6 antagonists, human TRPC6 antibodies and human TRPC6 antisense oligonucleotides are hereinafter alternatively referred to collectively as “agents of the invention”.

Neutrophil-associated inflammatory diseases to which the present invention is applicable include neutrophil-associated inflammatory or obstructive airways diseases, particularly chronic obstructive pulmonary disease (COPD), including chronic bronchitis and emphysema, and adult (or acute) respiratory distress syndrome (ARDS), rheumatoid arthritis and inflammatory bowel diseases such as Crohn's disease and ulcerative colitis. Eosinophil-associated inflammatory diseases to which the present invention is also applicable include eosinophil-associated inflammatory or obstructive airways diseases, particularly asthma and allergic rhinitis.

Amongst peripheral tissues, the lung along with the placenta has the highest level of TRPC6 expression. TRPC6 is present in many of the resident cells of the lung, for instance epithelial cells and airway smooth muscle cells, however TRPC6 is also present in smooth muscle in the pulmonary artery. TRPC6 is up-regulated in these smooth muscle cells when stimulated by platelet derived growth factor (PDGF). PDGF increases are associated with pulmonary hypertension and proliferation of artery smooth muscle cells. Inhibition of TRPC6 expression e.g. using antisense suppresses PDGF-stimulated pulmonary artery smooth muscle cell proliferation. Accordingly, pharmaceuticals that inhibit TRPC6 in smooth muscle in the pulmonary artery are useful in the treatment of pulmonary hypertension.

A human TRPC6 polynucleotide can be isolated using any suitable conventional method. Since the sequence for the human TRPC6 gene is known, specific primers may be used as a convenient option. For example, (SEQ 01) 5′-gcaaatgaaagctttggacc-3′ as the forward primer and (SEQ 02) 5′-atcgtaacattatagactccat-3′ as the reverse primer gives a polymerase chain reaction (PCR) product of 300 base pairs.

A human TRPC6 polynucleotide may be cDNA, genomic DNA or RNA and may be obtained using any suitable conventional method. For example it may be prepared from the nucleotides which it comprises by chemical synthesis, e.g. automated solid phase synthesis using known procedures and apparatus.

A human TRPC6 antibody may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11). Human TRPC6 antibodies may be used to detect, or determine the level of expression of, human TRPC6 polypeptides, or to inhibit ligand/anti-ligand binding activities of human TRPC6 polypeptides.

A human TRPC6 antisense oligonucleotide may be DNA, an analogue of DNA such as a phosphorothioate or methylphosphonate analogue of DNA, RNA, an analogue of RNA, or a peptide nucleic acid (PNA). The antisense oligonucleotide may be synthesised by conventional methods, for example using automated solid phase techniques. It may be used to inhibit the expression of the human TRPC6 gene, where this is desired. Alternatively, a short interfering RNA (siRNA) can be used as a specific tool for targeted gene knockdown. RNA interference inhibits gene expression and can therefore be used to explore gene function. This technique is described by S. M. Elbashir et al in Methods 26 (2002) 199-213.

A human TRPC6 polynucleotide probe comprises at least 15 contiguous nucleotides of the aforementioned polynucleotide or a complement thereof. The probe may be cDNA, genomic DNA or RNA and can be synthesised by conventional methods. Usually it is a synthetic oligonucleotide comprising 15 to 50 nucleotides, which can be labelled, e.g. with a fluorophore, to provide a detectable signal. The nucleotides that encode the first 50 or so amino acids of human TRPC6 may be used for this purpose. A human TRPC6 polynucleotide probe can be used to detect the presence or absence of the human TRPC6 gene, i.e. to detect genetic abnormality.

The effectiveness of an agent of the invention in inhibiting or reversing a leukocyte -associated inflammatory disease may be demonstrated in a model of the disease, e.g. a lipopolysaccharide-induced lung inflammation model in rat or mouse or models described by Durie et al., Clin. Immunol. Immunopathol. (1994) 73: 11-18; and Williams et al, Proc. Natl. Acad. Sci. USA (1992) 89: 9784-9788.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, one or more co-solvents known in the art such as ethanol (up to 20% by weight), one or more surfactants such as oleic acid or sorbitan trioleate, and one or more bulking agents such as lactose.

When the composition comprises a dry powder formulation, the active ingredient preferably has a particle diameter up to 10 microns and the formulation includes a diluent or carrier, such as lactose, and a compound that helps to protect against product performance deterioration due to moisture.

When the composition comprises a nebulised formulation, it preferably contains, for example, the active ingredient, which is either dissolved or suspended, in a vehicle containing water, a co-solvent such as ethanol or propylene glycol and a stabiliser, which may be a surfactant. The invention includes (i) an agent of the invention in inhalable form, e.g. in an aerosol or other atomizable composition or in inhalable particulate, e.g. micronised form, (ii) an inhalable medicament comprising an agent of the invention in inhalable form; (iii) a pharmaceutical product comprising such an agent of the invention in inhalable form in association with an inhalation device; and (iv) an inhalation device containing an agent of the invention in inhalable form.

Dosages of agents of the invention employed in practising the present invention may of course vary depending, for example, on the particular condition to be treated, the effect desired and the mode of administration. In general, suitable daily dosages for administration by inhalation are of the order of 1 μg to 10 mg/kg while for oral administration suitable daily doses are of the order of 0.1 mg to 1000 mg/kg.

The invention is illustrated by the following Examples.

### EXAMPLE 1

### Detection of Transcripts of the Human TRPC6 Gene in Human Leukocytes

Human leukocytes are purified from EDTA (2 mg/ml) anti-coagulated venous blood from healthy volunteers. Human lymphocytes are isolated from the mononuclear cell population obtained from the Ficoll-Hypaque gradients using magnetic cell sorting using CD4 MULTISOR™ microbeads (Miltenyi Biotec) according to the manufacturer's instructions. The T lymphocytes (CD45RO-negative) were further purified from this fraction using CD45RA microbeads and CD45RO microbeads (Miltenyi Biotec) according to the manufacturer's instructions and cultured for 7-14 days (in RPMI culture medium containing 100 U/ml IL-2, 5 μg/ml transferrin and 1 U/ml insulin) into Th-1 like or Th-2 like lymphocytes on plates coated with a mixture of anti-CD3 and anti-CD28 antibody, or only anti-CD28 antibody respectively.

Human granulocytes are purified from EDTA (2 mg/ml) anti-coagulated venous blood from healthy volunteers by density centrifugation through Ficoll-Hypaque, dextran sedimentation, followed by erythrocyte lysis with hypotonic saline. The preparation is typically >95% neutrophils. Human eosinophils are isolated from this leukocyte population by magnetic cell sorting using CD16 microbeads (Miltenyi Biotec) according to the manufacturer's instructions (>98% purity). The residual CD16-positive neutrophils are eluted from the magnetic columns and used as a neutrophil source (>99% purity).

Messenger RNA prepared from each leukocyte type is reverse-transcribed to cDNA and used as templates for polymerase chain reactions using (SEQ 01) 5′-gcaaatgaaagctttggacc-3′ as the forward primer and (SEQ 02) 5′-tcgtaacattatagactccat-3′ as the reverse primer. That primer pair is specific for the human TRPC6 gene and amplifies a 300 base pair PCR product. The TRPC6 gene amplification product is detected by analysing the PCR products on an agarose gel.

These results indicate that TRPC6 is present in leukocytes, i.e. the cells that are present in inflammed tissues.

All the molecular techniques used are conventional techniques that are described in Sambrook, J., Fritsch, E. F., Maniatis, T. (1989) Molecular Cloning: A Laboratory Manual, 2nd edition, Cold Spring Harbour Laboratory Press, New York.

The polymerase chain reaction is described in Saiki, R. K., D. H. Gelfand, S. Stoffel, S. J. Scharf, R. Higuchi, G. T. Horn, K. B. Mullis, and H. A. Erlich. “Primer-Directed Enzymatic Amplification of DNA with a Thermostable DNA Polymerase.” Science 239 (1988): 487-491. and in U.S. Pat. No. 4,883,195.

### EXAMPLE 2

### Assay Suitable for High-throughput Screening

An assay for screening candidate or test compounds is performed using cells stably-transfected with TRPC6 and expressing a functional TRPC6 channel e.g. the mammalian cell line HEK 293 which expresses an endogenous muscarinic acetylcholine receptor which can activate TRPC6 channels. The assay is performed on a 96-well FLIPR® (Fluorescence Imaging Plate Reader (Molecular Devices Corporation) using the Molecular Devices proprietary FLIPR® membrane potential assay kit (cat # R8034) to measure membrane depolarisation resulting from TRPC6-mediated Na⁺ influx. The specificity of this TRPC6-mediated response may be shown by demonstrating that it is not observed in TRPC6-expressing cells if Na⁺ is replaced in the extracellular buffer by the impermeant monovalent cation NMDG⁺ and that Na⁺ influx-dependent membrane depolarisation is not observed in control cells that have not been transfected with TRPC6. The TRPC6-expressing cells are seeded in 96-well black-walled flat-bottomed microplates in a plating volume of 100 μl and grown to confluence for the assay. To prepare the cells for the assay, the cell culture medium is removed and 100 μl of loading buffer is added to each well. The loading buffer consists of FLIPR® membrane potential dye diluted to the required assay concentration in a buffer composed of 140 mM-NaCl, 0.15 mM CaCl₂, 3.3 mM KH₂PO4, 1.2 mM MgCl₂, 10 mM D-glucose and 20 m M HEPES, at pH7.4 and containing 0.03 mM BAPTA-AM. The plates are incubated at 37° C. for 20 minutes prior to commencing the assay. For the detection of inhibitors of TRPC6, test agents are added to each well either before or after stimulation with the muscarinic acetylcholine receptor agonist carbachol, which activates the TRPC6 channel. The expected effect of an inhibitor would be to reduce the carbachol-stimulated increase in fluorescence. Activators of TRPC6 may be detected by substituting carbachol for a test agent, where activators will stimulate an increase in fluorescence.

### EXAMPLE 3

### Detection of 1 -[b-[3-(4-methoxyphenyl)propoxy]-4-methoxyphenethyl]-1H-imidazole.HCl

TRPC6 clone 14 cells pre-treated with 1-[b-[3-(4-methoxyphenyl)propoxy]-4-methoxyphenethyl]-1H-imidazole.HCl for 10 minutes are stimulated with carbachol (10 μM) in accordance with the assay described in Example 2. The fluorescence is measured prior to carbachol addition (minimum response, i.e. Min.) and when the carbachol-stimulated increase in fluorescence has reached a maximum (maximum response, i.e. Max.). It can be seen in table 1 below that the reduced fluorescence responses are observed in compound treated-cells compared to the control response in the absence of compound.

TABLE 1
Inhibition of TRPC6 ion channels by 1-[b-[3-(4-methoxy-
phenyl)-propoxy]-4-methoxyphen-ethyl]-1H-imidazol-HCl

| Compound | Mean fluorescence response | |
|---|---|---|
| concentration (μM) | calculated as (Max − Min)/Min | S.E.M. |
| 0 (i.e. Control) | 1.219 | 0.089 |
| 0.1 | 1.172 | 0.099 |
| 03 | 1.128 | 0.109 |
| 1.0 | 0.977 | 0.082 |
| 3.0 | 0.699 | 0.041 |
| 10.0 | 0.278 | 0.055 |
| 30.0 | 0.122 | 0.021 |

The data is calculated as a (Max−Min)/Min response. Each concentration of 1-[b-[3-(4-methoxyphenyl)propoxy]-4-methoxyphenethyl]-1H-imidazole.HCl is the mean±SEM (standard error of the mean) of n=10 wells. The calculated mean IC₅₀ value from n=4 experiments is 4.9±0.8 μM (mean±S.E.M.).

This shows that the assay described in Example 2 detects 1-[b-[3-(4-methoxyphenyl)propoxy]-4-methoxyphenethyl]-1H-imidazole.HCl.

1. A method of identifying a substance suitable for use in the treatment of a leukocyte-associated inflammatory disease which modulates the activity of a polypeptide encoded by the human transient receptor potential 6 gene, wherein the method comprises combining a candidate substance with said polypeptide and measuring the effect of the candidate substance on the activity of said polypeptide.

2. A method according to claim 1 wherein the candidate substance is combined with cells that are stably-transfected with TRPC6 and express a functional TRPC6 channel and measuring any membrane depolarisation to indicate a TRPC6-mediated Na+ influx.

3. A method according to claim 1 wherein the cells express an endogenous calcium-mobilising G protein-coupled receptor that can activate TRPC6 channels by agonist stimulation and a receptor agonist is added after the candidate substance to stimulate the TRPC6 channels and allow for any inhibition to be measured.

4. A pharmaceutical composition comprising a compound that inhibits the influx of calcium ions through a human TRPC6 ion channel and a pharmaceutically acceptable carrier.

5. A pharmaceutical composition according to claim 4 wherein the compound is 1-[b-[3-(4-methoxyphenyl)propoxy]-4-methoxyphenethyl]-1H-imidazole.HCl.

6. A method of treating a leukocyte-associated inflammatory disease in a subject in need of such treatment, which comprises administering to said subject an effective amount of an antibody which is immunoreactive with a polypeptide encoded by the human TRPC6 gene or an antisense oligonucleotide comprising a nucleotide sequence complementary to a polynucleotide comprising a nucleotide sequence encoding that polypeptide.

7. The method according to claim 6, in which the disease is a neutrophil-associated disease such as chronic obstructive pulmonary disease, adult respiratory distress syndrome, rheumatoid arthritis or inflammatory bowel disease.

8. The method according to claim 6, in which the disease is an eosinophil-associated disease such as asthma or allergic rhinitis.

9. A method of treating a leukocyte-associated inflammatory disease in a subject in need of such treatment, which comprises administering to said subject an effective amount of a TRPC6 inhibitor.

10. A method of treating pulmonary hypertension in a subject in need of such treatment, which comprises administering to said subject an effective amount of a TRPC6 inhibitor.
